(19) United States
(12) Patent Application Publication
(10) Pub. No.: US 2022/0301661 A1
(43) Pub. Date: Sep. 22, 2022

(54) METHOD AND SYSTEM FOR PREDICTING PRODUCT OF AROMATIC HYDROCARBON ISOMERIZATION PRODUCTION PROCESS

(71) Applicant: EAST CHINA UNIVERSITY OF SCIENCE AND TECHNOLOGY, Shanghai (CN)

(72) Inventors: Wenli DU, Shanghai (CN); Minglei YANG, Shanghai (CN); Weimin ZHONG, Shanghai (CN); Feng QIAN, Shanghai (CN); Zhi LI, Shanghai (CN)

(21) Appl. No.: 17/631,080

(22) PCT Filed: Dec. 2, 2020
(86) PCT No.: PCT/CN2020/133337

§ 371 Date: Jan. 28, 2022

(30) Foreign Application Priority Data
Feb. 10, 2020 (CN) 202010084365.2

(51) Int. Cl. G16C 20/30; G16C 20/10; G16C 20/70

(52) U.S. Cl. CPC G16C 20/30; G16C 20/10; G16C 20/70

(57) ABSTRACT

A method and system for predicting products of an aromatic hydrocarbon isomerization production process are provided. The method includes generating multiple initial sample points, using a mechanism the actual output response values of all of the initial sample points; establishing an radial basis function (RBF) model according to the initial sample points and actual output response values thereof; using a particle swarm optimization (PSO) algorithm to find expected deviation between nearest neighbors as well as the sampling point having the largest sparsity product, using mechanism model to calculate the output response value of said sampling point on RBF model, adding output response value into the sample points, and reconstructing the surrogate model; repeating the previous step until the upper limit for the number of sample points is reached to obtain a final RBF model; and using the RBF model to predict product yield of aromatic hydrocarbon isomerization.

### CROSS REFERENCE TO RELATED APPLICATION(S)

The present application claims priority to the Chinese Patent Application No. CN 202010084365.2, filed with the China National Intellectual Property Administration (CNIPA) on Feb. 10, 2020, and entitled “METHOD AND SYSTEM FOR PREDICTING PRODUCT OF AROMATIC HYDROCARBON ISOMERIZATION PRODUCTION PROCESS”, which is incorporated herein by reference in its entirety.

### TECHNICAL FIELD

The present disclosure relates to a prediction technology for yield of a key product (such as hydrogen yield) in an aromatics isomerization production link, and specifically, to a prediction technology for key performance indicators (such as yield information of key products) in an aromatics isomerization production process by describing an original industrial process using a surrogate model modeling based on a mechanism model.

### BACKGROUND ART

Large-scale industrial production of aromatics is realized through an advanced aromatics complex technology, the advanced aromatics complex technology has a complicated flowsheet, accompanied by a high conversion of para-xylene. C₈ aromatics isomerization and toluene disproportionation are two important aromatics conversion processes in the aromatics complex technology. The purpose of the isomerization is to convert o-xylene (OX), m-xylene (MX) and ethylbenzene (EB) to p-xylene (PX) which is of higher value among xylene isomers.

Xylene isomerization is mainly conducted by a reactor and a separation system. The flowsheet of Xylene isomerization is shown in FIG. 1. Reactor 1 is mainly used to conduct isomerization under the function of a catalyst to convert PX-poor C₈ aromatics mixture into an C₈ aromatics mixture close to thermodynamic equilibrium. In the separation system, the reaction product needs to achieve gas-liquid separation through separation tower 5 before distillation. Circulating hydrogen is mixed with supplementary hydrogen through compressor 4 and then mixed with C₈ aromatics (C₈A) in heat exchanger 3. The reactant mixture is further heated in heating furnace 2 and then enter the reactor 1. Liquid product is separated by the separation tower 5 enters a light component-removing rectification tower 6, and yield is increased through a reflux tank 7; a part of light component enters a circulation tower 8, liquefied gas is produced from a reflux tank 9, and heavy aromatics at the bottom of the circulation tower can be recycled.

The isomerization mainly includes reactions of isomerization of xylene and EB, and side reactions such as disproportionation, dealkylation and hydrocracking. By-products include benzene and trimethylbenzene produced by disproportionation of the xylene. Transalkylation between the EB and xylene can produce more by-products, including toluene, methyl-ethyl benzene, dimethyl ethyl benzene and the like. Almost no dealkylation occurs during the isomerization under normal operation. However, the EB may be dealkylated into benzene when the reaction reaches a certain temperature to reduce the yield of C₈ aromatics. In addition, the isomerization is conducted with cycloalkanes as a reaction intermediate. In the presence of hydrogen, a small amount of cycloalkane intermediates and hydrogen undergo a ring-opening cracking reaction to generate linear alkanes of different lengths, which affects the yield of C₈ aromatics and the rate of PX isomerization.

In the actual production, temperature, pressure, feed flow and other parameters of the isomerization may fluctuate intermittently or periodically due to an influence of upstream and downstream. Some parameters are sensitive to product quality, yield, energy consumption and the like, and improper operation can easily have a greater impact on the efficiency of the isomerization. The conventional mechanism model has relatively high accuracy in predicting the yield and optimizing calculation, but is difficult to meet real-time requirements of the installation due to complex structure and low efficiency. Therefore, it is necessary to establish a surrogate model that can accurately describe entire process characteristics and have high computing efficiency to support simulation and operation optimization of the aromatics isomerization industrial process.

### SUMMARY

The following provides a brief description of one or more aspects to provide a basic understanding of these aspects. This description is not a detailed description of all conceived aspects and is neither intended to designate key or decisive elements in all of respects nor to attempt to define a scope of any or all of the aspects. The sole purpose is to provide some concepts of one or more aspects in a simplified form to provide more detailed description later.

To solve the above problems, the present disclosure discloses a method and a system for predicting products of aromatic hydrocarbon isomerization production process. In the present disclosure, a surrogate model is established through a complete description of aromatics isomerization to correctly predict changes of a yield with feed and operation parameters based on the surrogate model, thereby ensuring product quality and stable operation of production process.

Technical solutions are as follows: the present disclosure reveals a method for predicting products of aromatic hydrocarbon isomerization production process, the method includes following steps.

In step 1, operation conditions of a specified aromatics isomerization production process are set as input variables of a surrogate model, yields of the specified aromatics isomerization production process are set as an output variables of the surrogate model, upper and lower limits of each input variable are set and multiple initial sample points are generated to form an initial sample set, and actual output response values of the multiple initial sample points are obtained through a mechanism model;

In step 2, a radial basis function (RBF) neural network surrogate model is established according to the initial sample points and the actual output response values of the initial sample points;

In step 3, a sampling point with a largest product of a nearest expected difference and a sparsity by using a particle swarm optimization (PSO) algorithm are found, and an output response value of the sampling point on the RBF neural network surrogate model is calculated by using the mechanism model, and the sampling point and the output response value are added to sample points to reconstruct the surrogate model;

In step 4, step 3 is repeated to continuously increase accuracy of the surrogate model until an upper limit of a number of the sampling points is reached, to obtain a final RBF neural network surrogate model; and

In step 5, an aromatics isomerization production process is simulated through the final RBF neural network surrogate model obtained, and the yields of aromatics isomerization product are predicted.

According to an example of the method, the operation conditions of the specified aromatics isomerization production process as the input variables of the surrogate model may include: isomerization feed, circulating hydrogen, supplementary hydrogen, isomerization reaction temperature, isomerization reaction pressure, EB content, MX content and OX content; and the yields of the specified aromatics isomerization production process as an output variable of the surrogate model may include: tail hydrogen yield, dry gas yield, light hydrocarbon yield and mixed C₈ yield.

According to an example of the method, in step 1, the initial sample points may be generated by using Latin hypercube sampling within the upper and lower limits of each input variable; and a sample set for testing may also be generated by using the Latin hypercube sampling in a search space.

According to an example of the method, in step 2, the initial sample points may be normalized before the RBF neural network surrogate model is established, and an initial RBF neural network surrogate model may be established according to these initial sample points by using a Cubic RBF.

According to an example of the method, the actual output response values of the initial sample point may be obtained by substituting the initial sample points into a Hysys mechanism model.

According to an example of the method, a new sampling point

$${x}_{new} = {arg}_{x ∈ R} max Sparsity ( x ) \times N E D ( x ) )$$

may be found by using the PSO algorithm in step 3, where Sparsity(x) represents a sparsity of a sampling point x, NED(x) represents a nearest expected difference of a sampling point x, R represents a sample space; and a product of the sparsity and the nearest expected difference is maximized to obtain a sample point x_{new} with a highest uncertainty.

The present disclosure further reveals a system for predicting products of aromatic hydrocarbon isomerization production process, the system includes:

a sample generation module, configured to set operation conditions of a specified aromatics isomerization production process as input variables of a surrogate model, set yields of the specified aromatics isomerization production process as output variables of the surrogate model, set upper and lower limits of each input variable and generate multiple initial sample points to form an initial sample set, and obtain actual output response values of the multiple initial sample points through a mechanism model;

an initial surrogate model establishment module, configured to establish an RBF neural network surrogate model according to the initial sample points and the actual output response values of the initial sample points;

a surrogate model reconstruction module, configured to find a sampling point with a largest product of a nearest expected difference and a sparsity by using a PSO algorithm, and calculate an output response value of the sampling point on the RBF neural network surrogate model by using the mechanism model, and add the sampling point and the output response value to sample points to reconstruct the surrogate model;

a final surrogate model establishment module, configured to repeat process of the surrogate model reconstruction module to continuously increase accuracy of the surrogate model until an upper limit of the number of the sampling points is reached, to obtain a final RBF neural network surrogate model; and

a model prediction module, configured to simulate an aromatics isomerization production process through the final RBF neural network surrogate model obtained, and predict the yields of an aromatics isomerization product.

According to an example of the system, in the sample generation module, the operation conditions of the specified aromatics isomerization production process as an input variables of the surrogate model may include: isomerization feed, circulating hydrogen, supplementary hydrogen, isomerization reaction temperature, isomerization reaction pressure, EB content, MX content and OX content; and the yields of the specified aromatics isomerization production process as an output variables of the surrogate model may include: tail hydrogen yield, dry gas yield, light hydrocarbon yield and mixed C₈ yield.

According to an example of the system, in the sample generation module, the initial sample points may be generated by using Latin hypercube sampling within the upper and lower limits of each input variable; and a sample set for testing may also be generated by using the Latin hypercube sampling in a search space.

According to an example of the system, in the initial surrogate model establishment module, the initial sample points may be normalized before the RBF neural network surrogate model is established, and an initial RBF neural network surrogate model may be established according to these initial sample points by using a Cubic RBF.

According to an example of the system, the actual output response values of the initial sample points may be obtained by substituting the initial sample points into a Hysys mechanism model.

According to an example of the system, a new sampling point

$${x}_{new} = {arg}_{x ∈ R} max Sparsity ( x ) \times N E D ( x ) )$$

may be found by using the PSO algorithm, where Sparsity(x) represents a sparsity of a sampling point x, NED(x) represents a nearest expected difference of the sample point x, R represents a sample space; and a product of the sparsity and the nearest expected difference is maximized to obtain a sample point x_{new} with a highest uncertainty.

Compared with the prior art, the present disclosure has the following beneficial effects:

1. The aromatics isomerization model is constructed by using the surrogate model. Compared with the conventional mechanism models, with a same input variable, an output of corresponding key performance indicators can be obtained in a very short time, with much higher calculation efficiency.

2. Greatly-reduced calculation time is conducive to real-time prediction and agile optimization of operation variables.

3. An area with the largest change and the largest uncertainty in the aromatics isomerization model can be found by looking for the point where a product of the sparsity and the nearest expected difference is the largest. In this way, the accuracy of the surrogate model can be greatly improved every time a new sampling point is added, such that the final surrogate model can have sufficient accuracy to replace the original mechanism model.

### BRIEF DESCRIPTION OF THE DRAWINGS

Foregoing features and advantages of the present disclosure will be better understood after reading a detailed description of the examples of the present disclosure with reference to following drawings. In the figures, components are not necessarily drawn to scale, and the components having similar related characteristics or features may have same or similar reference numerals.

FIG. 1 shows a schematic diagram in an aromatics isomerization production process.

FIG. 2 shows a flow chart of an example of a method for predicting products in an aromatics isomerization production process according to the present disclosure.

FIG. 3 shows a structural block diagram of an example of a system for predicting products in an aromatics isomerization production process according to the present disclosure.

FIGS. 4a to 4d show schematic diagrams of root-mean-square error (RMSE) variation curves of various yields of the aromatics isomerization surrogate model.

FIGS. 5a to 5d show schematic diagrams of prediction results of various yields of the aromatics isomerization surrogate model.

### DETAILED DESCRIPTION OF THE EMBODIMENTS

The present disclosure will be described in detail below with reference to accompanying drawings and specific examples. It should be noted that aspects described below in connection with the accompanying drawings and specific examples are merely examples, and are not to be construed as limiting the scope of the present disclosure.

The principle of the present disclosure is to establish a surrogate model through a mechanism model of an aromatics isomerization link, and continuously improve the accuracy of the constructed surrogate model by using adaptive sampling, and finally applying the resultant surrogate model to real-time prediction and optimization.

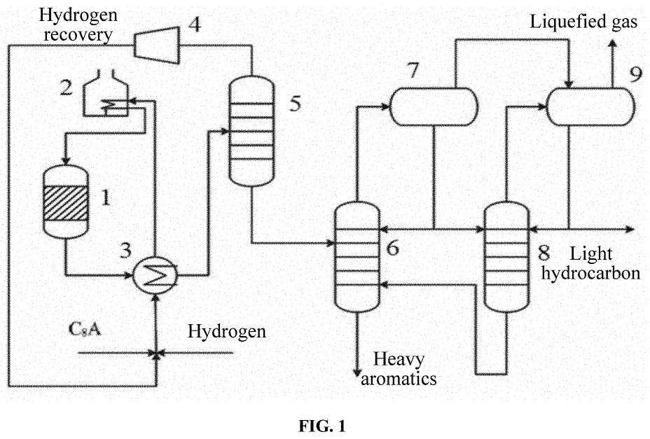

A process flow of the aromatics isomerization is shown in FIG. 1. An isomerization unit includes a reaction system, a separation system and a hydrogen circulation system. The isomerization unit mainly includes a heating furnace 2, a heat exchanger 3, a reactor 1, a separation tower 5, a fractionating column 6, a hydrogen compressor 4, reflux tanks 7 and 9, a circulation tower 8 and other equipment. After a raffinate from an adsorption separation device is mixed with circulating hydrogen and supplementary hydrogen, the resultant mixture enters the heating furnace 2 through the heat exchanger 3, is heated to reaction temperature and then enters the reactor 1 to conduct isomerization under a catalyst. A reaction product enters the separation tower 5 for gas-liquid separation. Most of discharged hydrogen is sent back to the reactor 1 through the compressor 4 for recycling, and liquid material enters the light component-removing fractionating column 6 to remove light component, and a yield is increased through the reflux tank 7. A part of the light component enters the circulation tower 8, liquefied gas is produced from the reflux tank 9, and heavy aromatics at the bottom of the circulation tower 8 can be recycled.

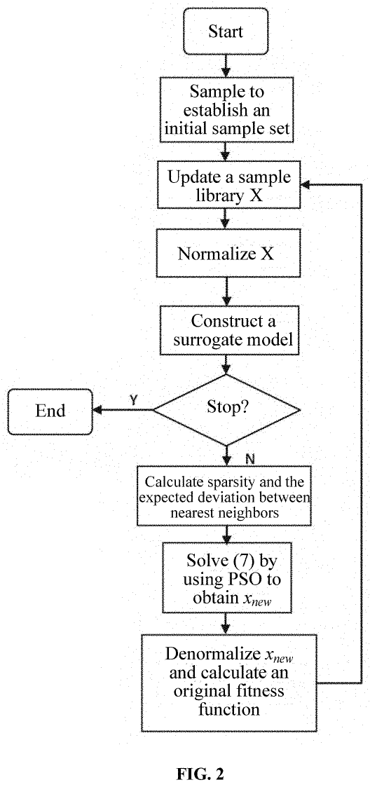

FIG. 2 shows a flow chart of an example of the method for predicting product of aromatic hydrocarbon isomerization production process according to the present disclosure. As shown in FIG. 2, a detailed description of implementation steps of the example is as follows.

In step 1, operation conditions of a specified aromatics isomerization production process are received as input variables of a surrogate model, yields of products of the specified aromatics isomerization production process are received as output variables of the surrogate model, upper and lower limits of each input variable are set and multiple initial sample points are generated to form an initial sample set, and actual output response values of the multiple initial sample points are obtained through a mechanism model, while a test sample set is randomly generated for an accuracy verification of the surrogate model.

In this step, operation conditions that have a greater impact on the reaction and product in the aromatics isomerization production link are generally selected as the input variables of the surrogate model, including: isomerization feed, circulating hydrogen, supplementary hydrogen, isomerization reaction temperature, isomerization reaction pressure, ethylbenzene (EB) content, m-Xylene (MX) content and o xylene (OX) content. The yields of the products of the aromatics isomerization production link are selected as the output variables of the surrogate model, including: tail hydrogen yield, dry gas yield, light hydrocarbon yield and mixed C₈ yield.

In an example, 8 operation conditions in the aromatics isomerization production link, including the isomerization feed, circulating hydrogen, supplementary hydrogen, isomerization reaction temperature, isomerization reaction pressure, EB content, MX content and OX content, are used as the input variables of the surrogate model, and the upper and lower limits [xₘᵢₙ^{j},xₘₐₓ^{j}], j=1, 2, . . . , d of these 10 input variables are set, and the Latin hypercube sampling is used to obtain 20 initial samples X=[x₁, x₂, . . . , xₙ,]^{T}, where xᵢ=[xᵢ¹, xᵢ², . . . , xᵢ^{d}], n represents the number of sample points, d represents the variable dimension. In this example, n=20 and d=8. The actual output response values of the product yields of the 20 initial samples are obtained by using a Hysys mechanism model (that is, the initial samples are substituted into the Hysys mechanism model to obtain the actual output response values), and the 20 initial samples constitute an initial sample set. Another test sample set of 100 samples are obtained by using the same method to detect the accuracy of a final surrogate model.

The 20 initial samples are normalized one by one to eliminate an influence of sample dimension on calculations:

$${\tilde{x}}_{i}^{j} = \frac{{x}_{max}^{j} - {x}_{i}^{j}}{{x}_{max}^{j} - {x}_{min}^{j}} {\tilde{x}}_{i}^{j} ∈ [ 0 , 1 ] \qquad ( 1 )$$

\tilde{x}ᵢ^{j} represents a normalized result of the j-th dimension of the i-th sample, xₘₐₓ^{j} represents the upper limit of the j-th dimension, xₘᵢₙ^{j} represents the lower limit of the j-th dimension, and xᵢ^{j} represents the j-th dimension of the i-th sample. The actual output response values [y₁, y₂, . . . , yₙ] of the product yields are obtained by using the Hysys model and the initial samples, thus an initial training sample set is obtained.

In step 2, an radial basis function (RBF) neural network surrogate model is established according to the initial sample points and the actual output response values of the initial sample points.

The above example is continued to be described.

An initial surrogate model is established. An expression of the RBF model is as follows:

$$\hat{y} ( x ) = {\sum }_{i = 1}^{n} {λ}_{i} ϕ ( {r}_{i} ) + p ( x ) \qquad ( 2 )$$

where, ∥.∥ represents an Euclidean norm, λ₁, λ₂, . . . , λₙ∈R represents a weight coefficient, ϕ is the RBF, rᵢ=∥x−xᵢ∥ is an Euclidean distance between a point x to be measured and the sampling point xᵢ, xᵢ=[xᵢ¹, xᵢ², . . . , xᵢ^{d}], n represents a number of sample points, d represents variable dimension, and p(x) is a polynomial, which may be expressed as xb+a, b=[b₁, b₂, . . . , bₙ]^{T}. Generally, common RBF ϕ is shown in Table 1, and Cubic RBF is used here.

TABLE 1
Commonly used RBFs

| | | | Thin plate | | | Inverse |
|---|---|---|---|---|---|---|
| | Linear | Cubic | spline | Gaussian | Multi-quadric | multi-quadric |
| ϕ | r | r³ | r²(ln)r | exp(−r²/c²) | ${( {r}^{2} + {c}^{2} )}^{\frac{1}{2}}$ | ${( {r}^{2} + {c}^{2} )}^{- \frac{1}{2}}$ |

Required parameters are calculated by the following formula:

$$[ \begin{matrix}Φ & p \\ {p}^{T} & 0\end{matrix} ] [ \begin{matrix}λ \\ c\end{matrix} ] = [ \begin{matrix}F \\ 0\end{matrix} ] , \\ where p = [ \begin{matrix}{x}_{1} \\ {x}_{2} \\ … \\ {x}_{n}\end{matrix} ] , λ = [ \begin{matrix}{λ}_{1} \\ {λ}_{2} \\ … \\ {λ}_{n}\end{matrix} ] c = [ \begin{matrix}{b}_{1} \\ {b}_{2} \\ … \\ {b}_{n} \\ d\end{matrix} ] , F = [ \begin{matrix}{y}_{1} \\ {y}_{2} \\ … \\ {y}_{n}\end{matrix} ] , \qquad ( 3 )$$

where Φ is a n×n matrix filled by Φ_{i,j}=ϕ(∥xᵢ−xⱼ∥), yᵢ(i=1, . . . , n) represents a true response value corresponding to the sampling point xᵢ. The matrix

$$[ \begin{matrix}Φ & p \\ {p}^{T} & 0\end{matrix} ]$$

is full rank, and a linear system has only one unique solution, such that a RBF surrogate model that uniquely describes a true objective function can be obtained.

The root-mean-square error (RMSE) of the surrogate model is calculated:

$$R M S E = \sqrt{\frac{1}{N} {\sum }_{i = 1}^{N} ( {y}_{i} - {\hat{y}}_{i} )} \qquad ( 4 )$$

In the formula (4), yᵢ and ŷᵢ are a true response value and a surrogate model response value at i-th test point, respectively, and N is a number of test points.

In step 3, a sampling point with a largest product of a nearest expected difference and a sparsity is found by using a Particle Swarm Optimization (PSO) algorithm, and an output response value of the sampling point on the RBF neural network surrogate model is calculated by using the mechanism model, and the sampling point and the output response value are added to the sample point set to reconstruct the surrogate model.

The above example is continued to be described.

The definition of sparsity in this example is as follows:

sampling points X=[x₁, x₂, . . . , xₙ]^{T} are existed, n represents the number of sample points, the upper and lower limits of the search space are UP={up₁, up₂, . . . , up_{d}} and DOWN={down₁, down₂, . . . , down_{d}}. d represents a number of dimensions, and the sparsity at any point x_{new} in the search space is defined as follows.

In Step 1, an Euclidean distance diatance between the new sampling point x_{new} and the existing sampling point X=[x₁, x₂, . . . , xₙ]^{T} is calculated, and sorting is conducted to obtain a diatance^{sort}.

In Step 2, X₂=[x₁, x₂, . . . , xₙ, x_{new}]^{T}

In Step 3, for j=1:D, [x₁^{j}, x₂^{j}, . . . , xₙ^{j}, x_{new}^{j}] is sorted from small to large to obtain [x₁^{j}, x₂^{j}′, . . . , xₙ^{j}′, x_{new}^{j}′], that is, values of the i-th dimension are sorted from small to large to find a location pos where x_{new,j} is positioned. The distance diatance is sorted in the same way to obtain the diatance^{sort}.

if pos=1

In the sparsity of the i-th dimension, a lower limit is the lower limit sparsityᵢ^{down} (x_{new})=downᵢ of the sampling space, and an upper limit is the value sparsityᵢ^{sp}(x_{new})=xₖ_{₁}_{,i}′ of the i-th dimension of the point closest to x_{new} among the points larger than the x_{new,i} in the i-th dimension, where k₁=arg min(diatanceⱼ^{sort})(j=2, 3, . . . , n+1),

else if pos=n+1

In the sparsity of the i-th dimension, an upper limit is the upper limit sparsityᵢ^{up}(x_{new})=upᵢ of the sampling space, and a lower limit is the value sparsityᵢ^{down}(x_{new})=xₖ_{₂}_{,i}′ of the i-th dimension of the point closest to x_{new} among the points smaller than the x_{new,i} in the i-th dimension, where k₁=arg min(diatanceⱼ^{sort})(j=1, 2, . . . , n),

else

In the sparsity of the i-th dimension, an upper limit is the value sparsityᵢ^{down}(x_{new})=xₖ_{₁}′ of the i-th dimension of the point closest to x_{new} among the points larger than the x_{new,i} in the i-th dimension, where k₁=argmin(diatanceⱼ^{sort})(j=pos+1, pos+2, . . . , n+1); and a lower limit is the lower limit of the sampling space, that is, the value sparsityᵢ^{down}(x_{new})=xₖ_{₂}′ of the i-th dimension of the point closest to x_{new} among the points smaller than the x_{new,i}, where k₂=arg min(diatanceⱼ^{sort})(j=1, 2, . . . , pos−1).

end

In Step 4, sparsity Sparsity(x_{new}) corresponding to the x_{new} is finally obtained:

$$Sparsity ( {x}_{n e w} ) = {\prod }_{i = 1}^{D} ( {sparsity}_{i}^{u p} - {sparsity}_{i}^{d o w n} ) \qquad ( 5 )$$

The nearest expected difference (NED) is defined as follows: there are sampling points X={x₁, x₂, . . . , xₙ}, the corresponding actual outputs are Y={y₁, y₂, . . . , yₙ}, and a surrogate model ŷ(x) is constructed according to X and Y. The nearest expected difference NED(x_{new}) of the point x_{new} is:

NED(x_{new})=(ŷ(x_{new})−yₙₑₐᵣₑₛₜ)² (6)

yₙₑₐᵣₑₛₜ is corresponding true response value of the point closest to the x_{new} in the distance X={x₁, x₂, . . . , xₙ}, and ŷ(x_{new}) is the response value of x_{new} on the surrogate model. A larger NED(x_{new}) leads to a larger approximate gradient near the new sampling point x_{new}, that is, the function fluctuates greatly and needs to focus on sampling.

Then, a new sampling point is found according to the maximum product of the sparsity and the nearest response value difference.

$${\tilde{x}}_{new} = {arg}_{x ∈ R} max Sparsity ( x ) \times N E D ( x ) ) \qquad ( 7 )$$

The sparsity Sparsity(x) is responsible for controlling a global search, and the nearest expected difference NED(x) is responsible for a local key information search. R is a sampling space domain. \tilde{x}_{new} is found by using the PSO algorithm, where the PSO algorithm has a number of iterations which is set to 100, and a population size which is set to 25.

Finally, the obtained \tilde{x}_{new} is denormalized.

$${x}_{n e w}^{j} = \frac{1 - {\tilde{x}}_{n e w}^{j}}{1 - 0} ( {x}_{max}^{j} - {x}_{min}^{j} ) j = 1 , 2 , … , d \qquad ( 8 )$$

x_{new}^{j} represents a denormalized result of the j-th dimension \tilde{x}_{new}^{j} of the \tilde{x}_{new}, xₘₐₓ^{j} represents the upper limit of the j-th dimension, and xₘᵢₙ^{j} represents the lower limit of the j-th dimension. Actual key performance indicators y_{new} corresponding to x_{new} are calculated by using the Hysys model. The x_{new} and y_{new} are added into the training sample set.

In Step 4, step 3 is continuously repeated to continuously increase accuracy of the surrogate model until an upper limit of the number of sampling points is reached, to obtain a final RBF surrogate model.

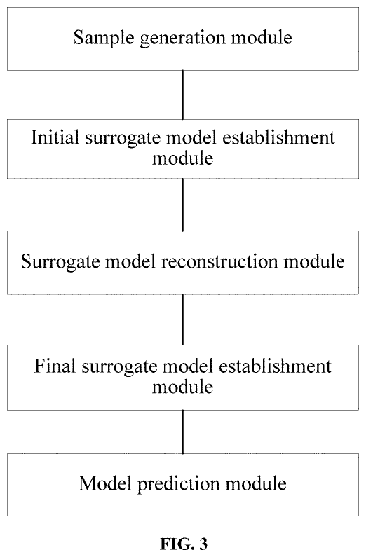

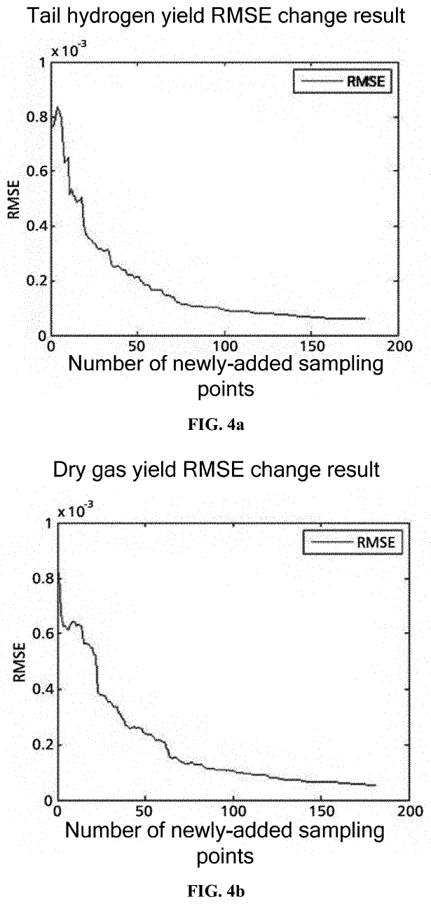

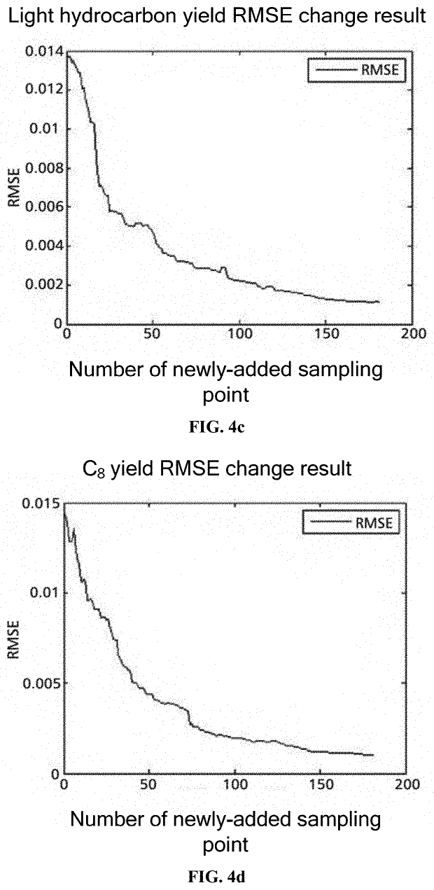

The above example is continued to be described. There are 20 initial sample points, and the upper limit of the number of evaluations is 180. The above steps are repeated for the four yields to establish four RBF surrogate models. The RMSE of the four RBF surrogate models varies with the number of iterations, and their results are shown in FIGS. 4a to 4d. It can be seen that the accuracy of the entire model continues to increase with the increase in the number of sampling points, and the final RBF surrogate model has errors within an acceptable range, and can be used to accurately predict the product.

In Step 5, an aromatics isomerization production process is simulated through the established RBF surrogate model, and the yield of an aromatics isomerization product is predicted.

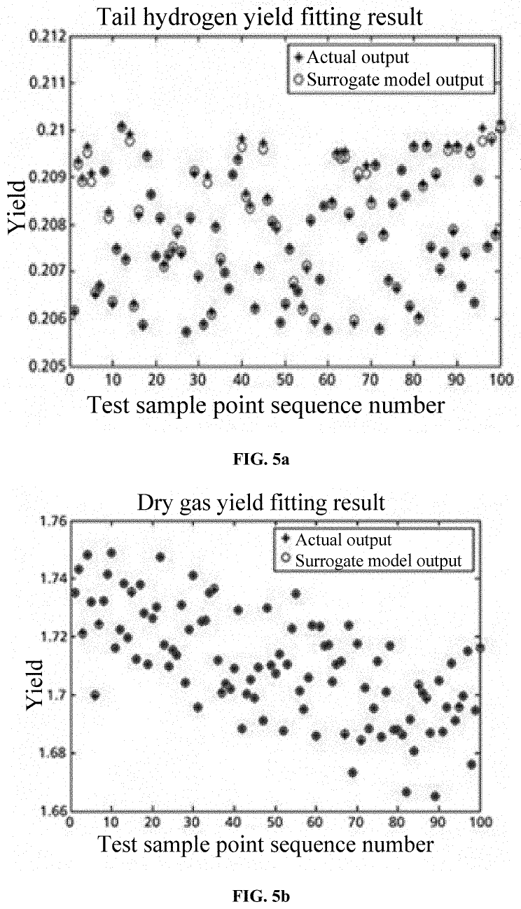

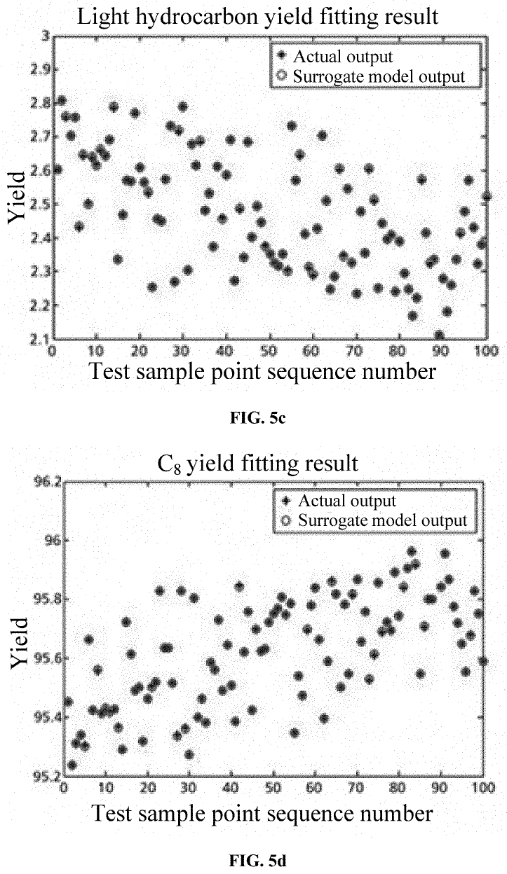

The above example is continued to be described, results of the final model on the test sample are shown in FIGS. 5a to 5d. It can be seen that the surrogate model is very accurate in predicting the four key performance indicators.

FIG. 3 shows a principle of an example of the system for predicting product of aromatic hydrocarbon isomerization production process according to the present disclosure. As shown in FIG. 3, the system of the example includes: a sample generation module, an initial surrogate model establishment module, a surrogate model reconstruction module, a final surrogate model establishment module and a model prediction module.

The sample generation module is configured to set operation conditions of a specified aromatics isomerization production process as input variables of a surrogate model, set yields of the specified aromatics isomerization production process as an output variables of the surrogate model, set upper and lower limits of the input variables and generate multiple initial sample points to form an initial sample set, and obtain actual output response values of the multiple initial sample points through a mechanism model.

In the sample generation module, the initial sample points are generated by using Latin hypercube sampling within the upper and lower limits of each input variable; and the sample set for testing are also generated by using the Latin hypercube sampling in a search space.

In the sample generation module, the operation conditions of a specified aromatics isomerization production process as input variables of a surrogate model includes: isomerization feed, circulating hydrogen, supplementary hydrogen, isomerization reaction temperature, isomerization reaction pressure, EB content, MX content and OX content; and the yields of the specified aromatics isomerization production process as output variables of the surrogate model includes: tail hydrogen yield, dry gas yield, light hydrocarbon yield and mixed C₈ yield.

The initial surrogate model establishment module is configured to establish an RBF neural network surrogate model according to the initial sample points and the actual output response values of the initial sample points. The actual output response values of the initial sample points are obtained by substituting the initial sample points into a Hysys mechanism model.

In the initial surrogate model establishment module, the initial sample points are normalized before the RBF neural network surrogate model is established, and an initial RBF neural network surrogate model is established according to these initial sample points by using a Cubic RBF.

The surrogate model reconstruction module is configured to find sampling points with a largest product of a nearest expected difference and a sparsity by using a PSO algorithm, and calculate output response values of the sampling points on the RBF neural network surrogate model by using the mechanism model, and add the sampling points and the output response values to the sample points to reconstruct the surrogate model.

A new sampling point

$${x}_{new} = {arg}_{x ∈ R} max Sparsity ( x ) \times N E D ( x ) )$$

is found by using the PSO algorithm in the surrogate model reconstruction module, where Sparsity(x) represents the sparsity of the sampling point x, NED(x) represents the nearest expected difference; and a product of the sparsity and the nearest expected difference is maximized to obtain a sample point x_{new} with a highest uncertainty.

The final surrogate model establishment module is configured to repeat processes of the surrogate model reconstruction module to continuously increase accuracy of the surrogate model until an upper limit of the number of sampling points is reached, to obtain a final RBF neural network surrogate model.

The model prediction module is configured to simulate an aromatics isomerization production process through the established RBF neural network surrogate model, and predict the yields of an aromatics isomerization product.

The foregoing description of the present disclosure is provided to enable any person skilled in the art to make or use the present disclosure. Various modifications to this disclosure will be apparent to those skilled in the art, and the generic principles defined herein may be applied to other variations without departing from the spirit or scope of the present disclosure. Therefore, the present disclosure is not intended to be limited to the examples and designs described herein, but should be granted the widest range consistent with the principles and novel features disclosed herein.

1. A method for predicting products of an aromatic hydrocarbon isomerization production process, comprising the following steps:
a setting step for setting operation conditions of a specified aromatics isomerization production process as input variables of a surrogate model, setting yields of the specified aromatics isomerization production process as output variables of the surrogate model, setting upper and lower limits of each input variable and generating multiple initial sample points to form an initial sample set, and obtaining actual output response values of the multiple initial sample points through a mechanism model;
an establishing step for establishing a radial basis function (RBF) neural network surrogate model according to the initial sample points and the actual output response values of the initial sample points;
a finding step for finding a sampling point with a largest product of a nearest expected difference and a sparsity by using a particle swarm optimization (PSO) algorithm, and calculating an output response value of the sampling point on the RBF neural network surrogate model by using the mechanism model, and adding the sampling point and the output response value to sample points to reconstruct the surrogate model;
a repeating step for repeating step 3 to continuously increase accuracy of the surrogate model until an upper limit of a number of the sampling points is reached, to obtain a final RBF neural network surrogate model; and
a simulating step for simulating an aromatics isomerization production process through the final RBF neural network surrogate model obtained, and predicting the yields of aromatics isomerization products.

2. The method for predicting products of an aromatic hydrocarbon isomerization production process according to claim 1, wherein the operation conditions of the specified aromatics isomerization production process as the input variables of the surrogate model comprises: isomerization feed, circulating hydrogen, supplementary hydrogen, isomerization reaction temperature, isomerization reaction pressure, ethylbenzene (EB) content, m-xylene (MX) content and o-xylene (OX) content; and the yields of the specified aromatics isomerization production process as the output variable of the surrogate model comprises: tail hydrogen yield, dry gas yield, light hydrocarbon yield and mixed C₈ yield.

3. The method for predicting products of an aromatic hydrocarbon isomerization production process according to claim 1, wherein in setting step, the initial sample points are generated by using Latin hypercube sampling within the upper and lower limits of each input variable; and a sample set for testing is also generated by using the Latin hypercube sampling in a search space.

4. The method for predicting products of an aromatic hydrocarbon isomerization production process according to claim 1, wherein in establishing step, the initial sample points are normalized before the RBF neural network surrogate model is established, and an initial RBF neural network surrogate model is established according to these initial sample points by using a Cubic RBF.

5. The method for predicting products of an aromatic hydrocarbon isomerization production process according to claim 1, wherein the actual output response values of the initial sample points are obtained by substituting the initial sample points into a Hysys mechanism model.

6. The method for predicting products of an aromatic hydrocarbon isomerization production process according to claim 1, wherein a new sampling point
$${x}_{new} = {arg}_{x ∈ R} max Sparsity ( x ) \times N E D ( x ) )$$
is found by using the PSO algorithm in the finding step, Sparsity(x) represents a sparsity of a sampling point x, NED(x) represents a nearest expected difference of a sampling point x, R represents a sample space; and a product of the sparsity and the nearest expected difference is maximized to obtain a sample point x_{new} with a highest uncertainty.

7. A system for predicting products of an aromatic hydrocarbon isomerization production process, comprising:
a sample generation module, configured to set operation conditions of a specified aromatics isomerization production process as input variables of a surrogate model, set yields of the specified aromatics isomerization production process as output variables of the surrogate model, set upper and lower limits of each input variable and generate multiple initial sample points to form an initial sample set, and obtain actual output response values of the multiple initial sample points through a mechanism model;
an initial surrogate model establishment module, configured to establish an RBF neural network surrogate model according to the initial sample points and the actual output response values of the initial sample points;
a surrogate model reconstruction module, configured to find a sampling point with a largest product of a nearest expected difference and a sparsity by using a PSO algorithm, and calculate an output response value of the sampling point on the RBF neural network surrogate model by using the mechanism model, and add the sampling point and the output response value to sample points to reconstruct the surrogate model;
a final surrogate model establishment module, configured to repeat process of the surrogate model reconstruction module to continuously increase accuracy of the surrogate model until an upper limit of the number of the sampling points is reached, to obtain a final RBF neural network surrogate model; and
a model prediction module, configured to simulate an aromatics isomerization production process through the final RBF neural network surrogate model obtained, and predict the yields of an aromatics isomerization product.

8. The system for predicting products of an aromatic hydrocarbon isomerization production process according to claim 7, wherein in the sample generation module, the operation conditions of the specified aromatics isomerization production process as the input variables of the surrogate model comprises: isomerization feed, circulating hydrogen, supplementary hydrogen, isomerization reaction temperature, isomerization reaction pressure, EB content, MX content and OX content; and the yields of the specified aromatics isomerization production process as the output variables of the surrogate model includes: tail hydrogen yield, dry gas yield, light hydrocarbon yield and mixed yield.

9. The system for predicting products of an aromatic hydrocarbon isomerization production process according to claim 7, wherein in the sample generation module, the initial sample points are generated by using Latin hypercube sampling within the upper and lower limits of each input variable; and a sample set for testing is also generated by using the Latin hypercube sampling in a search space.

10. The system for predicting products of an aromatic hydrocarbon isomerization production process according to claim 7, wherein in the initial surrogate model establishment module, the initial sample points are normalized before the RBF neural network surrogate model is established, and an initial RBF neural network surrogate model is established according to these initial sample points by using a Cubic RBF.

11. The system for predicting products of an aromatic hydrocarbon isomerization production process according to claim 7, wherein the actual output response values of the initial sample points are obtained by substituting the initial sample points into a Hysys mechanism model.

12. The system for predicting products of an aromatic hydrocarbon isomerization production process according to claim 7, wherein a new sampling point
$${x}_{new} = {arg}_{x ∈ R} max Sparsity ( x ) \times N E D ( x ) )$$
is found by using the PSO algorithm, Sparsity(x) represents a sparsity of a sampling point x, NED(x) represents a nearest expected difference of the sampling point x, R represents a sample space; and a product of the sparsity and the nearest expected difference is maximized to obtain a sample point x_{new} with a highest uncertainty.
